(19) United States
(12) Patent Application Publication
(10) Pub. No.: US 2010/0036325 A1
(43) Pub. Date: Feb. 11, 2010

(54) Medical Needle Safety Device

(76) Inventor: Barry Peter Liversidge, Essex (GB)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 12/442,139

(22) PCT Filed: Sep. 21, 2007
(86) PCT No.: PCT/GB2007/050569

§ 371 Date: Oct. 27, 2009

(30) Foreign Application Priority Data
Sep. 22, 2006 (GB) 0618699.3
Sep. 21, 2007 (GB) PCT/GB2007/050569

(51) Int. Cl. A61M 5/32

(52) U.S. Cl. 604/198

(57) ABSTRACT

A safety device for a medical needle having a sharp tip and a mount spaced from the tip includes a tubular support for the needle mount and a protective sleeve carried by the support for sliding movement between a retracted position and a protecting position. A slot is formed in the sleeve with portions of greater width at the forward and rearward ends of the slot. The tubular support has a guide element slidably received in the slot and a locking element deformable inwardly of the sleeve and receivable in the greater width portions of the slot. The locking element is profiled to permit forward sliding movement of the sleeve from its retracted position but when received in the rearward portion of the slot, then resists rearward sliding movement of the sleeve.

This invention relates to a safety device for a medical needle having a sharp tip and a mount spaced from the tip and intended for penetration of a human or animal body, or for other medical uses such as the penetration of a pierceable membrane of an intravenous medication system. The invention further relates to the combination of a safety device as aforesaid and a housing therefor, and to the combination of a safety device and a medical needle. For convenience, in the following all medical uses of the needle will be described simply as the penetration of a body, even though specific embodiments may be intended for other medical uses.

Throughout this specification the terms forward and forwardly used in relation to the safety device and a syringe refer to the end thereof which is approached to a body when a procedure is to be performed, and the direction towards that end. Conversely, the terms rearward and rearwardly refer to the end of the safety device opposed to the forward end and the direction away from that forward end.

Fluids of various kinds may be administered to a human or animal body by means of a hollow needle in conjunction with a source of the required fluid. For example, such a needle may be used in conjunction with a syringe holding a liquid drug which may be contained directly in the syringe barrel or in a cartridge located within the syringe, the needle being used to penetrate the body at the site at which the drug is to be received. Equally, body fluids may be withdrawn by using a hollow needle which is used to penetrate the body until the tip is located at the site from which fluid is to be withdrawn.

A recognised hazard for clinicians and other persons using medical needles for the above described purposes, as well as people who may be exposed to used needles in the course of the disposal of those needles, is the risk of a so-called needle-stick injury—that is to say the accidental penetration of a person's skin by the needle. Prior to the use of the needle to supply a fluid to or to withdraw fluid from a body, this rarely presents much of a problem, though once the needle has been used on a body, there is a very much higher risk of a serious consequence for a person suffering a needle-stick injury. During use of the needle to penetrate the body tissues of a patient, the needle is likely to become contaminated with various organisms; should a person subsequently suffer a needle-stick injury, infection could occur.

There have been numerous proposals for protecting the sharp tip of a used needle, in order to reduce the risk of a needle-stick injury following use of the needle. Some proposals have actually increased the likelihood of such an injury by virtue of the action which must be performed to protect the tip, even if the risk thereafter is lessened. Despite all of the proposals which have previously been made, very few have achieved commercial success, nor has there been wide acceptance by the medical industry. Many proposals are somewhat complex and involve a significantly greater manufacturing cost, and so are unacceptable on economic grounds. Others are much more difficult to use as compared to an unprotected needle, and so are rejected by clinicians. Yet further proposals do not allow compliance with best practice protocols.

A device which protects a needle tip after use without an operator having to perform any extra step on withdrawing the needle from a body is usually referred to as a passive protection device. This may be contrasted with an active protection device, where an operator is required to perform an extra step in order to protect a needle, following the withdrawal of the needle from a body. The requirement to perform an extra step leaves the needle unprotected for a longer period than with a passive protection device but conversely an active device may be much simpler in concept, easier to manufacture and more reliable in use. Further, an active protection device may also be significantly smaller than a passive device, in that an active device usually has fewer components, and so may be more acceptable to clinicians as being less obtrusive, when in use.

Health and safety legislation has led to a significant demand for protection devices for use with medical needles and which allow a clinician or perhaps others to use the needle in much the same way as is done with an unprotected needle, but which can be manufactured economically and which provide a high degree of protection against needle-stick injury. Particularly for self-injection, the protection device should be simple in concept, unobtrusive in use and easy and reliable to operate. So far, no widely accepted active protection device is available and which meets these requirements.

The present invention aims at providing a safety device advantageously in the form of an accessory for an injector such as a so-called pen injector primarily intended for self-administration of a medicament, which addresses these issues and which is both relatively simple and economic to manufacture, especially on a fully automated production line, and which does not significantly affect the performance of a conventional injection procedure when mounted on an injector.

According to this invention, there is provided a safety device for a medical needle having a sharp tip and a mount spaced from the tip, which device comprises:
a tubular support adapted for direct or indirect association with the needle mount;
a protective sleeve disposed co-axially on the support for sliding movement with respect thereto from a retracted position where a part of the length of an associated needle back from its tip is exposed, and then to a protecting position where the sleeve covers the needle;
a slot in the sleeve and extending substantially parallel to the sleeve axis which slot has portions of greater width at the forward and rearward ends thereof;
a locking member formed on the support and comprising a guide element slidably received in the slot and a locking element resiliently deformable inwardly of the sleeve and receivable in said portions of the slot, the locking element being profiled to permit forward sliding movement of the sleeve from its retracted position but to resist rearward sliding movement of the sleeve when in its protecting position.

It will be appreciated that the safety device of this invention is an active protection device, in that following the performance of an injection, the clinician or user of a syringe with which the device is associated has to slide the protective sleeve forwardly of the syringe until that protective sleeve reaches a protecting position, where the sleeve is locked against subsequent rearward movement to expose the needle. When the device is first fitted to a medical needle, which may or may not be attached at that point to a syringe, the sleeve is in its initial position with the locking element in the forward slot portion of greater width than the narrower part of the slot interconnecting said slot portions, and the guide element at least partially in the narrower part of the slot. Subsequent to performing the injection, the sleeve is pushed forwardly to its protecting position and when there, the locking element preferably locates in the rearward slot portion of greater width than the narrower part of the slot and thereby restrains subsequent rearward movement of the sleeve.

The portions of greater width of the slot, at the forward and rearward ends thereof, preferably extend circumferentially in both directions around the sleeve and thus to each side of the narrower part of the slot. For such a slot, the locking member preferably has a pair of locking elements of essentially the same form, arranged one to each side of the guide element whereby each locking element will be received in a respective increased width portion of the slot, with the protective sleeve both at its retracted (initial) position and at its protecting position.

The safety device may have a plurality of circumferentially-spaced slots with a like number of locking members on the support. In a preferred embodiment, there are two such slots though only one need be provided or more than two could be provided.

The guide and locking elements of the (or each) locking member advantageously are formed integrally and are resiliently deformable as a unit towards of the axis of the sleeve to permit the locking element to slide along the inner surface of the sleeve from the forward portion of the slot to the rearward portion thereof. For such an arrangement, the guide element should have a sufficient radial height to permit the guide element to remain in the slot, notwithstanding the radially inward deformation of the locking member when the locking element runs along the inner surface of the sleeve.

The support may have a flange around the rearward end thereof, which flange supports the (or each) locking member so as to project forwardly from the flange, generally parallel to the axis of the support. The locking member may be resiliently deformable in the radial direction by deformation of the locking member, or of the flange in the vicinity of the junction between the locking member and the flange.

The tubular support preferably is adapted for engagement with the mount of the needle, in such a way that once engaged, it is difficult to remove the tubular support from said mount. Locking teeth may be provided within the tubular support, to assist in this. Further, it is preferred for the tubular support to engage the mount of the needle in such a way that once the mount of the needle is fully received in the support, rotation of the support turns the mount to permit unscrewing of a needle from a syringe.

The safety device may be provided with a medical needle having a sharp tip and a mount formed as an internally threaded hub for attachment to a syringe such as a pen injector and the tubular support of the safety device being engaged with the hub. In this way, the needle may be screw-threaded on to an externally threaded boss provided at the forward end of the syringe, by grasping and rotating the protective sleeve. In the alternative, the needle mount of the needle may be formed integrally with the tubular support, which support is internally profiled for engagement with a syringe.

This invention extends to the combination of a safety device of this invention as described above and a housing therefor, which housing is closed at one end, has a greater length than the safety device and is open at its other end to permit insertion and removal of the safety device. Such a combination may be provided as a sterile package, with said other end being sealed by a removable closure. Alternatively, the housing may have an opening at one end for receiving the protective sheath of a needle which opening is sized frictionally to grip the protective sheath of a needle. Fitting the safety device to a medical needle will engage the sheath with the opening and then removing the housing from the needle will strip the protective sheath from the needle.

An alternative form of housing is a relatively loose sliding fit over the safety device but is retained thereon until an injection is to be performed, by means of a releasable interconnection between the locking member and the housing. Such a housing may be provided with a tab to assist the pulling away of the housing from the safety device but as the housing is a loose sliding fit over the safety device, that device will not be triggered by forward movement of the safety device sleeve.

By way of example only, certain specific embodiments of safety device of this invention, for use with a medical needle and syringe, will now be described in detail, reference being made to the accompanying drawings in which:—

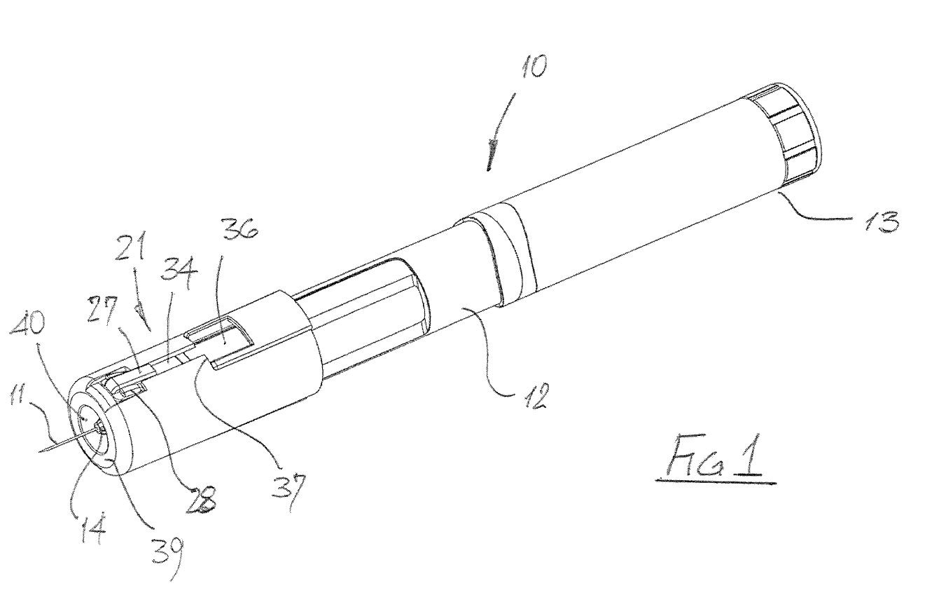

FIG. 1 is an isometric view of a syringe and needle fitted with a first embodiment of safety device of this invention, with the syringe ready for use;

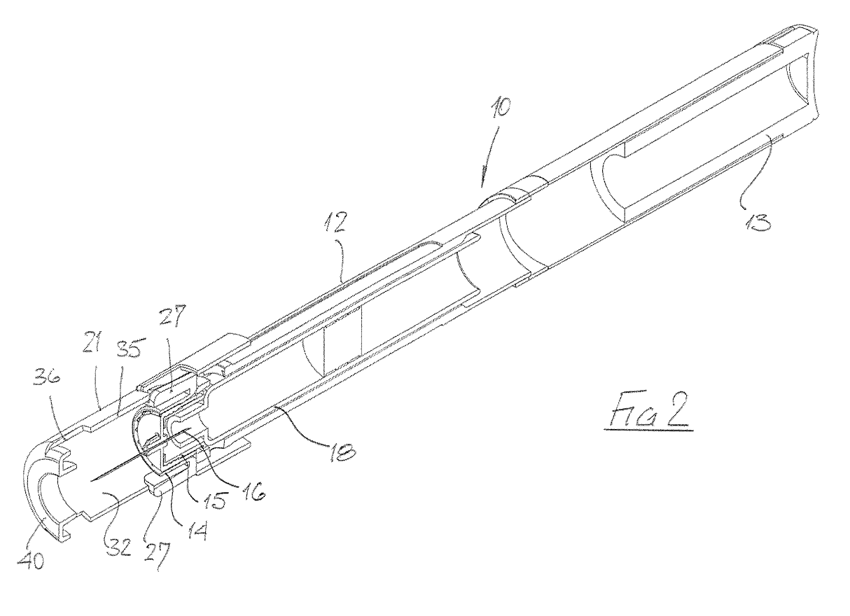

FIG. 2 is an axial sectional view through the syringe and safety device of FIG. 1, but on an enlarged scale and with the sleeve locked out in a protecting position;

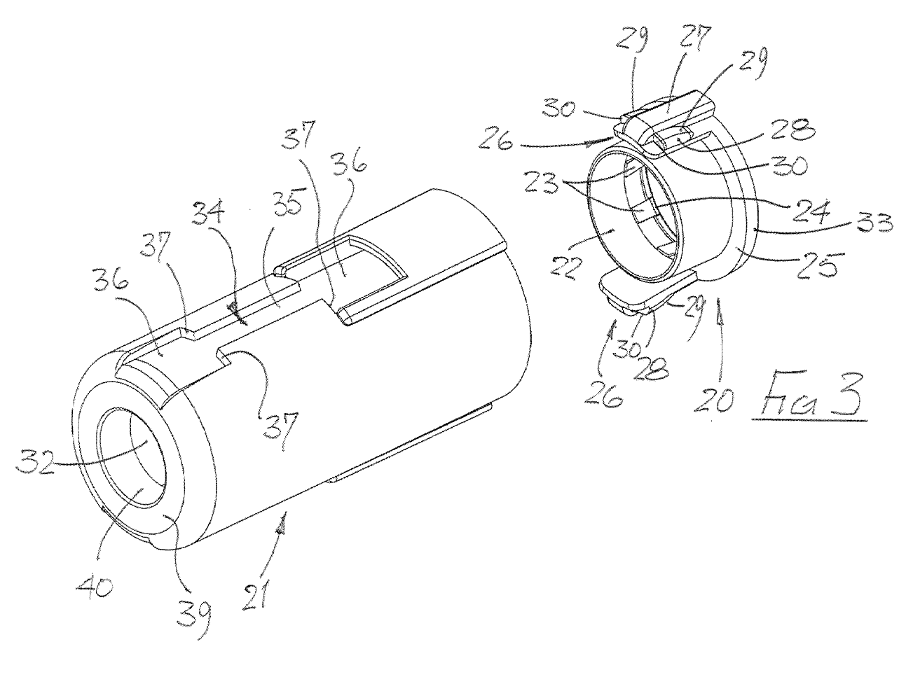

FIG. 3 is an exploded isometric view on an enlarged scale of the safety device shown in FIG. 1;

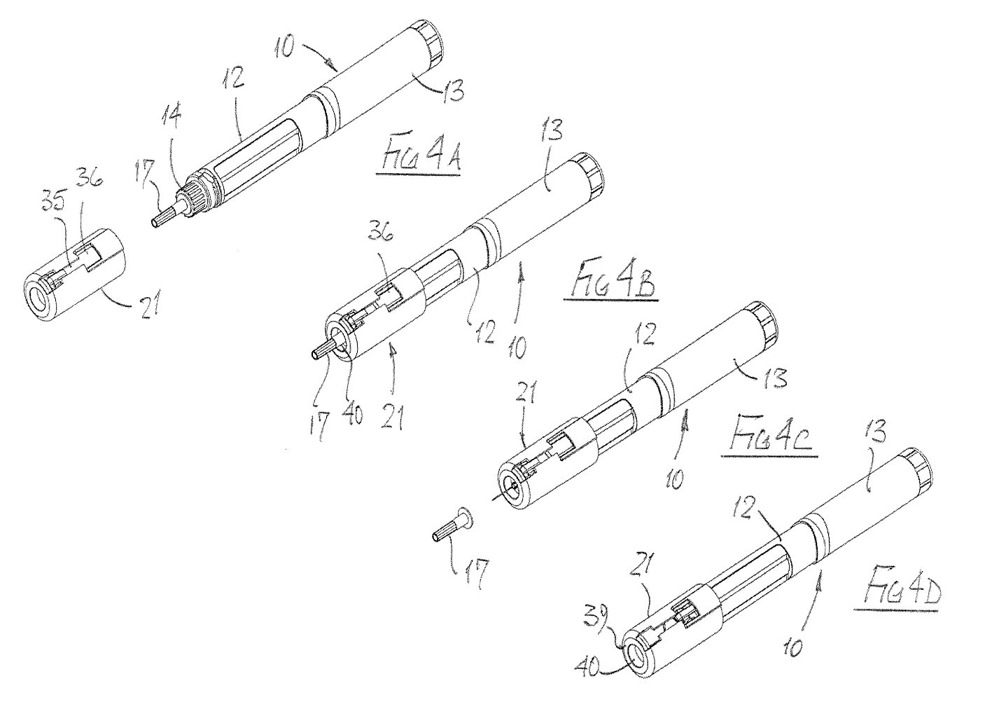

FIGS. 4A, 4B, 4C and 4D show the steps of using the safety device of FIG. 1, the illustration of the syringe and device of FIG. 4C corresponding to that of FIG. 1;

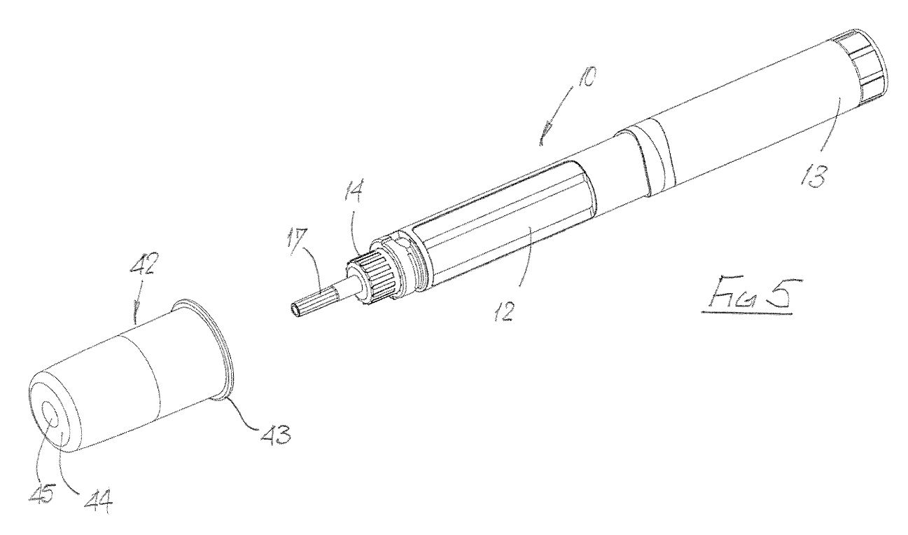

FIG. 5 is an isometric view of a syringe fitted with a sheathed needle and ready to receive a safety device corresponding to that of FIGS. 1 to 3 but provided within a housing;

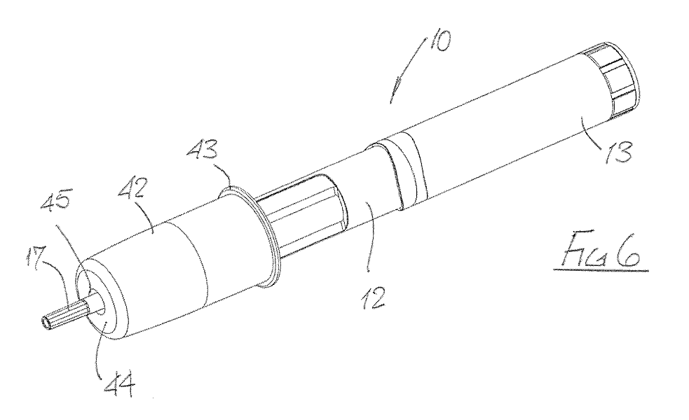

FIG. 6 shows the syringe and safety device of FIG. 5, with the safety device fitted to the syringe;

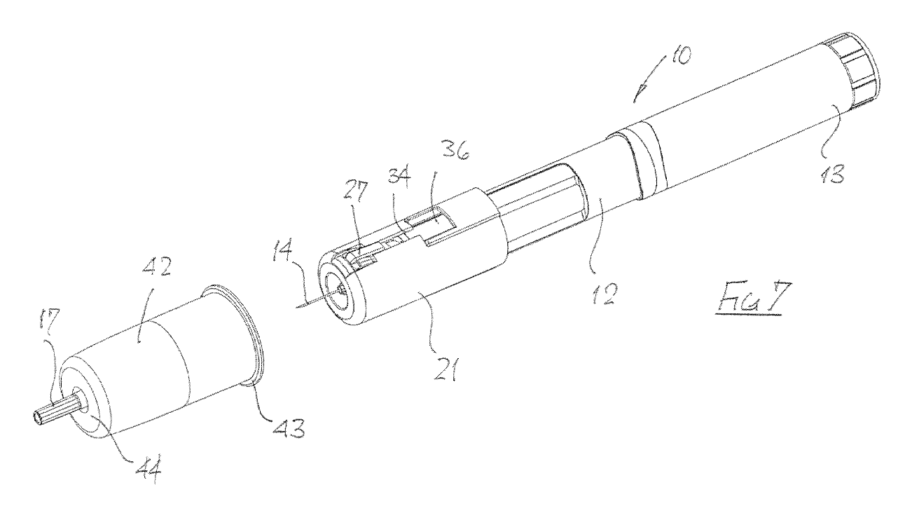

FIG. 7 shows the syringe and safety device of FIG. 6 but with the housing and sheath removed from the syringe, leaving the safety device connected thereto;

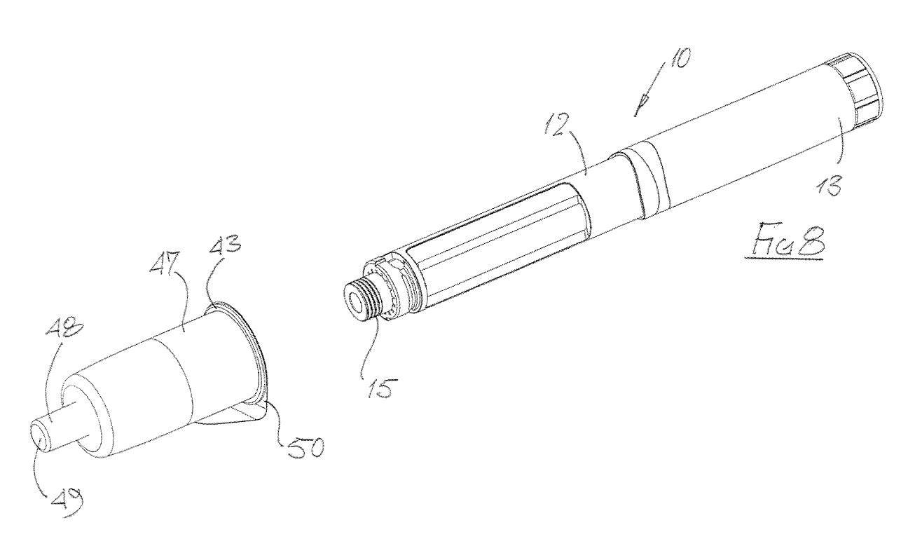

FIG. 8 shows a packaged safety device of this invention and including a needle, ready to be fitted to a syringe;

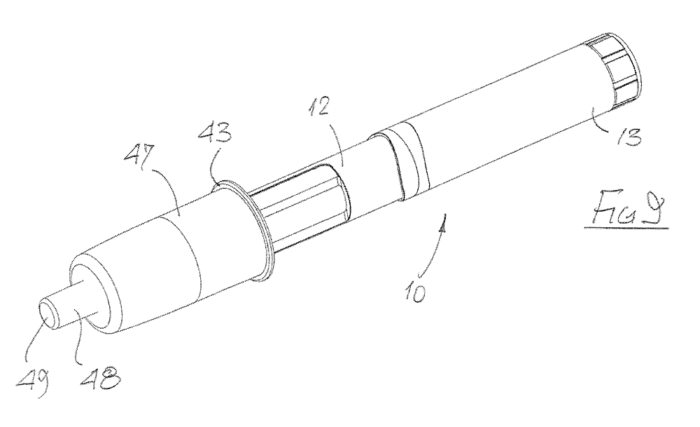

FIG. 9 shows the packaged safety device of FIG. 8 fitted to the syringe after removal of the package, the syringe and safety device then being as shown in FIG. 1;

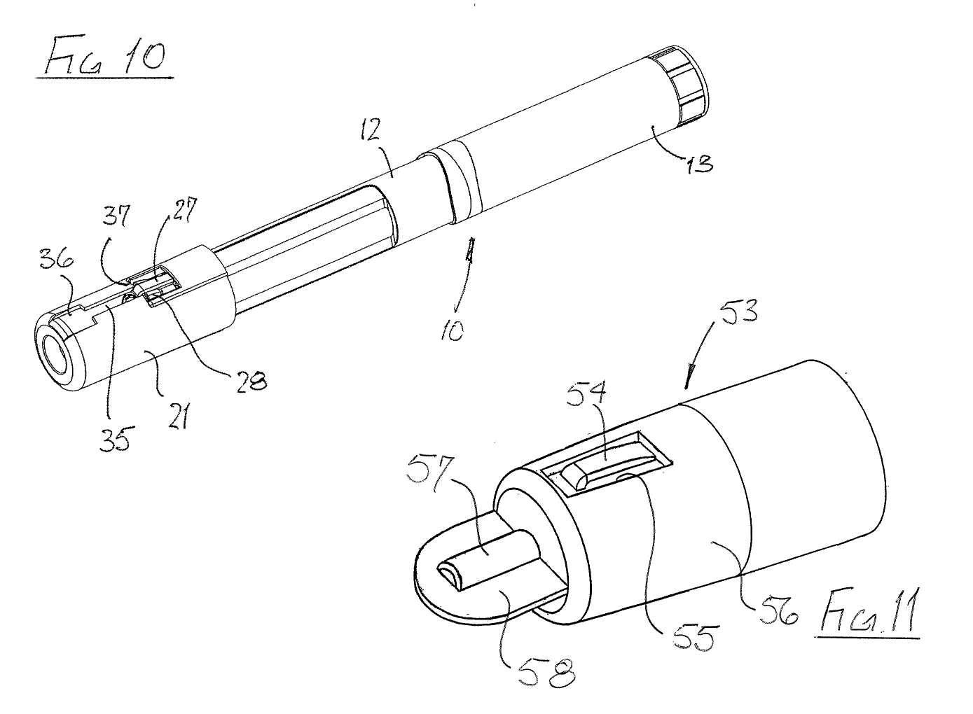

FIG. 10 shows the syringe and safety device of FIGS. 8 and 9 (but also of FIGS. 1 to 3) with the safety device locked out in a protecting position; and

FIG. 11 shows an alternative form of housing for the safety device.

Referring initially to FIGS. 1 to 4, there is shown a syringe 10 of the pen-injector variety, arranged for dispensing a pre-set dose of medicament out of a needle 11, from a vial of the medicament loaded into the syringe body 12. The syringe 10 includes a plunger having a head 13 which may be rotated for setting the required dose, the head moving rearwardly of the syringe (to the right in FIG. 1) during this setting, whereafter on depressing the head 13 the required dose is dispensed. Such a syringe is primarily intended for self-administration of the medicament and is very widely used especially for dispensing insulin, in the case of diabetes. The syringe per se is well known in the art and forms no part of this invention; consequently it will not be discussed in further detail here.

The needle 11 is carried in an internally threaded hub 14 (FIGS. 2 and 4A) which is screw-threaded to an externally threaded stub 15 provided at the forward end of the syringe body 12. The needle projects forwardly from the hub for performing an injection but is double-ended, the rearward part 16 of the needle penetrating a bung (not shown) provided at the forward end of the vial 18 (FIG. 2) of medicament disposed within the syringe. As supplied for use, the needle 11 is fitted with a relatively hard plastics material sheath 17 serving the combined purpose of protecting a user from an accidental needle-stick injury when fitting the hub 14 to the stub 15, and also protecting the needle (and especially its tip) from damage and contamination.

The safety device of this invention and intended for use with a syringe 10 and needle 11 comprises a tubular support 20 and a protective sleeve 21 slidably mounted on the support 20. The tubular support (see particularly FIGS. 2 and 3) has a bore 22 provided at its rearward end with a series of axially-extending ribs 23 equi-spaced around the internal circumference of the bore 22. The rearward end of each of those ribs includes a barb-shaped tooth 24 projecting by a small extent into the bore. The arrangement of the bore 22, the ribs 23 and teeth 24 is such that the support 20 may be pressed on to the outer surface of the needle hub 14, but when so received, it is very difficult to remove the support from the hub. Further, relative rotational movement between the support 20 and hub 14 is essentially impossible with all normal forces, once the support has been received fully on the hub.

At the rearward end of the support 20, there is an externally projecting flange 25 having a pair of diametrically opposed forwardly projecting arms 26. Each arm has a central guide 27 projecting radially outwardly but having a significant axial extent. To each side of the guide 27 there is provided a locking element 28 having a generally rounded rearwardly directed upper surface 29 and a generally radial abutment surface 30 at its forward end. Each arm is shown in FIG. 3 in its normal condition but each may be resiliently deflected radially inwardly of the tubular support 20.

The protective sleeve 21 is generally tubular in form and has an internal cylindrical surface 32 which is slidably received on the outer surface 33 of the flange 25. A pair of diametrically opposed slots 34 is formed through the wall of the sleeve 21, each slot extending essentially parallel to the axis of the sleeve 21 and having a narrower part 35 and end regions 36 of greater width than the narrower part 35. As best seen in FIG. 3, at each end region the slot 34 is enlarged to each side of the narrower part, so as to define a shoulder 37 at the junction between the narrower part and the respective end region.

The external surface of the protective sleeve 21 has an increased thickness in the vicinity of the rearward end regions of the slots 34, which facilitates handling of the device by holding the sleeve 21. At the forward end of the sleeve, there is formed an inwardly directed flange 39 having a central opening 40.

The use of the safety device will now be described, with particular reference to FIGS. 4A to 4D. The syringe is prepared in an entirely conventional manner, by selecting an appropriate needle and then screw-threading its hub 14 to the stub 15 at the forward end of the syringe body 12 (FIG. 4A). During this procedure the sheath 17 of the needle is left in place, again in accordance with the conventional procedure.

As supplied, the safety device has the protective sleeve 21 mounted on its tubular support 20, with each central guide 27 disposed partially in the narrower part 35 of the respective slot 34, and the two locking elements 28 disposed in the increased width end regions 36 at the forward ends of the slots 34. The safety device is then offered to the syringe and is pushed fully rearwardly such that the hub 14 of the needle is received fully within the bore 22 of the tubular support and is gripped by the ribs 23 and by the teeth 24 (FIG. 4B). The syringe is then prepared for performing an injection by pre-setting the required dose and pulling the sheath 17 away from the needle 11 (FIGS. 1 and 4C, though in these drawings the head of the plunger is shown against the syringe body 12 and so before pre-setting of the dose).

Following performance of an injection, the user manually pushes the protective sleeve 21 fully forwardly of the syringe, from the rearward end of that sleeve and so with no risk of a needle-stick injury. During the initial stage of movement of the sleeve from the position shown in FIGS. 1 and 4B, the rounded upper surface 29 of each locking element 28 serves as a cam acting against the shoulder 37, in order to facilitate radially inward deflection of the respective arm 26, the central guide 27 remaining in slot 34 notwithstanding this inward deflection. During continued forward movement of the sleeve, the guide 27 remains in the slot 34, to guide the sleeve and prevent relative rotation between the sleeve and the support 20. When the sleeve is moved fully forwardly to its protecting position (FIGS. 2 and 4D), the locking elements 28 are aligned with the rearward end region 36 of the slots 34, so allowing the arms 26 to spring outwardly once more. This results in the abutment surfaces 30 of the locking elements 28 confronting the shoulders 37 of the rearward end regions 36, which thus resist rearward movement of the sleeve, once more. Further, the sides of the locking elements 28 engage the sides of the rearward end regions 36, to prevent relative rotation between the sleeve 21 and tubular support 20

When the sleeve 21 has been locked in its protecting, forward position, the sleeve may be rotated counter-clockwise (viewed from the front). The support 20 is correspondingly rotated in view of the interengagement of the sides of the locking elements 28 with the rearward end regions 36 of the slots 34, and the interengagement of the support 20 with the needle hub serves to unscrew that hub 14 from the stub 15. When free, the needle together with the safety device may be thrown away in a safe manner, such as in a sharps box. Since the needle is not re-sheathed, the risk of needle-stick injury present during re-sheathing is eliminated.

FIGS. 5 to 7 show a syringe 10 fitted with a needle having a hub 14 and sheath 17, as described above, together with a safety device as described above but located within a tubular housing 42. The housing has an external flange 43 at its rearward end and an inward flange 44 at its forward end, which flange 44 has a central opening 45 for receiving a sheath 17 in a frictionally-engaging manner. The safety device contained within the housing is as described above with reference to FIGS. 1 to 3 and so will not be described again here. The housing 42 is profiled internally such that the protective sleeve of the device is a loose fit therewithin.

The housing 42 containing the safety device is presented to the syringe fitted with a needle (FIG. 5) and is pushed fully rearwardly (to the right in FIG. 5) so that the needle hub 14 is received wholly within the support, as described above with reference to FIGS. 1 to 3. The sheath 17 of the needle projects through the central opening 45 in flange 44 and is lightly gripped thereby such that on subsequently pulling the housing away from the protective sleeve 21 (FIG. 7), the sheath 17 is pulled away with the housing 42, so de-sheathing the needle 11. The syringe and safety device are, in this condition, corresponding to FIG. 1 and the procedure from this point is as described above.

FIGS. 8 to 10 show a packaged safety device including a needle pre-fitted therewithin and sealed in a sterile condition. The package has a tubular housing 47 within which the safety device is received, much as has been described above with reference to FIGS. 5 to 7. In this case, the housing includes a forwardly projecting tubular extension 48 closed at its forward end 49. Further, a strippable seal 50 is bonded to the flange 43 at the rearward end of the housing.

During manufacture of the packaged safety device, a needle and hub generally as described above is fitted into the bore 22 of the tubular support 20. Since the needle is wholly protected by the housing 47 and extension 48, there is no need for a sheath to be provided on that needle. The assembly is then sterilised and closed by seal 50.

In order to use the packaged safety device of FIGS. 8 to 10, the seal 50 is stripped from the housing 47 so exposing the needle hub 14. A syringe body 12 is offered to that needle hub and threaded thereto (FIG. 9), following removal of the seal 50 from the housing 47. After that, the housing 47 is pulled away from the safety device, whereafter the needle is exposed and the syringe is ready for use, as shown for example in FIG. 7. Once an injection has been performed, the sleeve is manually slid forwardly to its protecting position (FIG. 10), as with the other embodiments.

In all of the above embodiments, the arms 26 include a central guide 27 and a locking element 28 to each side of the central guide, which locking elements are of essentially the same form. One locking element of each arm could be replaced by a rounded camming surface which comes into engagement with shoulder 37 to one side of the associated slot 34 before the sleeve 21 has moved fully forwardly to its protecting position. In view of the resilience of the arms, that rounded camming surface may serve to ensure the sleeve continues to move forwardly until the abutment surface 30 engages behind shoulder 37, even should the forward force applied to the sleeve fall away, or perhaps be removed altogether.

In the above described embodiments, the slot 34 as well as the end portions of greater width are shown as open through the wall of the protective sleeve 21. However, so long as the wall thickness of the sleeve is sufficiently great, the slots could open solely inwardly of the sleeve and be closed radially outwardly. In such an arrangement access to the locking elements would be more difficult.

FIG. 11 shows an alternative housing 53, to replace either housing 42 or housing 47, described above. The safety device for use with housing 53 is a relatively loose fit within the internal bore of the housing. The safety device is retained within the housing by interaction between the central guide 54 of the tubular support 20 and an opening 55 formed in the wall 56 of the housing 53. Central guide 54 corresponds to central guide 27 of the embodiments described above (see for example FIG. 3) but is connected to the arm 26 only at the forward end thereof, so as to be capable of springing inwardly, out of the opening 55. In the alternative, and depending on the precise configuration, materials and so on, central guide 54 could be essentially the same as central guide 27, with reliance being placed on the resilience of the alternative housing 53 to allow the central guide to come free of the opening 55 in the housing. Another possibility would be to employ the locking element 28 for engagement in the opening 55, rather than the central guide 54, since that locking element is resiliently deformable inwardly of the sleeve. Though not shown in FIG. 11, a corresponding arrangement is provided diametrically opposite to the opening 55.

The forward end of the housing 53 includes a tube 57 corresponding to extension 48 (FIG. 8) adapted to receive the sheath of a needle and to strip that sheath from the needle when the housing 53 is pulled away from the needle. Around that tube 57 is a manually grippable tab 58, provided for the purpose of pulling the housing 53 away from a safety device located within the housing.

It will be appreciated that this alternative housing 53 holds the safety device within the housing by virtue of the interaction between the central guide 54 and the opening 55 in the housing wall 56. The housing may be pulled away from the locking member without pulling forwardly the protective sleeve of the safety device, since the protective sleeve is only a loose sliding fit within the housing. The needle sheath is stripped from the needle during this action.

1. A safety device for a medical needle having a sharp tip and a mount spaced from the tip, which device comprises:
a tubular support adapted for direct or indirect association with the needle mount;
a protective sleeve disposed co-axially on the support for sliding movement with respect thereto from a retracted position where a part of the length of an associated needle back from its tip is exposed, and then to a protecting position where the sleeve covers the needle;
a slot in the sleeve and extending substantially parallel to the sleeve axis which slot has portions of greater width at the forward and rearward ends thereof;
a locking member formed on the support and comprising a guide element slidably received in the slot and a locking element resiliently deformable inwardly of the sleeve and receivable in said portions of the slot, the locking element being profiled to permit forward sliding movement of the sleeve from its retracted position but to resist rearward sliding movement of the sleeve when in its protecting position.

2. A safety device as claimed in claim 1, wherein the locking element locates in the slot portion of greater width at the rearward end of the slot when the sleeve is in its protecting position.

3. A safety device as claimed in claim 2, wherein the portions of greater width of the slot at the forward and rearward ends thereof extend circumferentially in both directions around the sleeve and the locking member has a pair of locking elements arranged one to each side of the guide element.

4. A safety device as claimed in claim 2, wherein the sleeve is provided with a plurality of circumferentially-spaced slots and there is a like number of locking members on the support.

5. A safety device as claimed in claim 4, wherein the sleeve has two diametrically opposed slots of substantially the same shape there being two locking members on the support.

6. A safety device as claimed in claim 1, wherein the support has a flange around the rearward end thereof which flange supports the or each locking member so as to project forwardly generally parallel to the axis of the support.

7. A safety device as claimed in claim 6, wherein the guide and locking elements of the locking member are formed integrally and are resiliently deformable towards the axis of the sleeve to permit the locking element to slide along the inner surface of the sleeve, from the forward portion of the slot to the rearward portion thereof.

8. A safety device as claimed in claim 1, wherein the locking element has a rearwardly facing rounded profile and a forwardly facing abutment surface for engaging a shoulder defined by the rearward slot portion of greater width and which receives said locking element.

9. A safety device as claimed in claim 8, wherein there is a camming element on the opposite side of the guide element to the locking element, which camming element is receivable in the rearward slot portion and serves to urge the sleeve forwardly as the sleeve approaches its protecting position, thereby to cause the abutment surface of the locking element to engage said shoulder.

10. A safety device as claimed in claim 1, wherein the tubular support is adapted for engagement with the needle mount.

11. A safety device as claimed in claim 10, wherein the internal surface of the tubular support is formed with axially-extending ribs engageable with corresponding formations on the needle mount adapted for screw-threaded attachment to a syringe.

12. A safety device as claimed in claim 1 together with a medical needle having a sharp tip and a mount spaced from the tip, the mount being formed as an internally threaded hub for attachment to a syringe and the tubular support of the safety device being engaged with the hub whereby the needle is screw-threaded on to a syringe by grasping and rotating the protective sleeve.

13. A safety device as claimed in claim 1, wherein the needle mount is formed integrally with said tubular support and said tubular support is internally profiled for engagement with a syringe.

14. A safety device as claimed in claim 13, wherein said tubular support is internally threaded, for threading to an externally threaded boss formed at the forward end of a syringe.

15. In combination, safety device as claimed in claim 12 and a housing therefor, said housing being closed at one end, and having a greater length than is the safety device and needle, and said housing being open at its other end to permit insertion and removal of the safety device, said other end being sealed by a removable closure.

16. The combination of claim 15, wherein said closure comprises a strippable foil or sheet-like sterile barrier sealed to the end of the housing.

17. In combination, a safety device as claimed in claim 12 and a housing therefor, said housing having a greater length than the safety device and needle, and said housing having one end adapted to receive the forward end of the safety device, the housing loosely receiving the safety device and being releasably connected to the tubular support of said device.

18. The combination of claim 17, wherein the guide element of the locking member is releasably received within an opening in the housing when the safety device is fully received within the housing.

19. The combination of claim 18, wherein the one end of the housing is provided with a forwardly projecting manually grippable tab for pulling the housing away from the safety device.

20. The combination of claim 12, wherein the medical needle is provided with a protective sheath adapted for removal prior to use of the needle to perform an injection.

21. The combination of claim 20, wherein said one end of the housing has a forwardly projecting tube within which the sheath is received, the internal configuration of the tube being such that the sheath is frictionally gripped by the tube whereby removal of the housing from the combination de-sheaths the needle leaving the sheath at least partly within the tube.

22. The combination of claim 20, wherein the said one end of the housing has an opening for receiving the protective sheath of the needle and is open at its other end to permit insertion and removal of the safety device, the opening at said one end frictionally gripping the protective sheath so that fitting of the safety device to a medical needle and then removing the housing strips the protective sheath from the needle.
